(19) United States
(12) Patent Application Publication
(10) Pub. No.: US 2006/0030615 A1
(43) Pub. Date: Feb. 9, 2006

(54) Progesterone receptor modulators comprising pyrrole-oxindole derivatives and uses thereof

(75) Inventors: Andrew Fensome, Wayne, PA (US); Casey Cameron McComas, Phoenixville, PA (US); Edward George Melenski, Collegeville, PA (US); Michael Anthony Marella, Limerick, PA (US); Jay Edward Wrobel, Lawrenceville, NJ (US)

Correspondence Address: HOWSON AND HOWSON;CATHY A. KODROFF, ONE SPRING HOUSE CORPORATE CENTER, BOX 457, SPRING HOUSE, PA, 19477, US

(73) Assignee: Wyeth, Madison, NJ (US)

(21) Appl. No.: 11/175,457

(22) Filed: Jul. 6, 2005

Related U.S. Application Data
(60) Provisional application No. 60/599,900, filed on Aug. 9, 2004.

(51) Int. Cl. A61K 31/404

(52) U.S. Cl. 514/414

(57) ABSTRACT

Pyrrole-oxindole derivatives useful as progesterone receptor antagonists are provided. Pharmaceutical compositions containing these derivatives are described, as is the use thereof in contraception and hormone-related conditions.

### CROSS-REFERENCE TO RELATED APPLICATIONS

This application claims the benefit under 35 USC 119(e) of prior U.S. Provisional Patent Application No. 60/599,900, filed August 9, 2004.

### BACKGROUND OF THE INVENTION

Progesterone receptor (PR) agonists and antagonists, also termed PR modulators, have been described for use in contraception and a variety of other indications.

U.S. Pat. No. 6,562,857B2 describes compounds that are PR agonists. The genus is characterized by compounds of the formula: 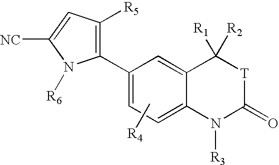
in which T is O, or absent; R₁, and R₂ are each, independently, hydrogen, alkyl, substituted alkyl or R₁ and R₂ are taken together to form a ring and together contain —CH₂(CH₂)ₙCH₂—; n=0-5; R₃ is hydrogen; R₄ is hydrogen or halogen; R₅ is hydrogen or alkyl; R₆ is hydrogen or alkyl; or a pharmaceutically acceptable salt thereof.

What are needed are novel PR modulators useful as contraceptives without the requirement for a progestin agonist or estrogen agonist.

### SUMMARY OF THE INVENTION

The compounds of this invention are progesterone receptor modulators which have utility in contraception and a variety of other applications. This PR antagonist mode of action offers advantages in contraception where the compound may be administered without co-administration of a progestin agonist or estrogen agonist and is free of the side effects of these agents.

In one embodiment, the compounds of the invention where R₉ in formula I is a C₁-C₆ alkyl, a C₁-C₄ alkyl, or methyl, exhibit the advantage of good potency.

Other aspects and advantages of the present invention are described further in the following detailed description of the preferred embodiments thereof.

### DETAILED DESCRIPTION OF THE INVENTION

The present invention provides compositions containing compounds of formula I: 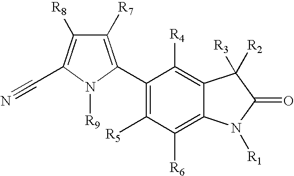
R₁ is hydrogen, alkyl, substituted alkyl, cycloalkyl, C₃-C₆ alkenyl, or C₃-C₆ alkynyl;
R₂ and R₃ are independently selected from among hydrogen, alkyl or substituted alkyl, or R₂ and R₃ are taken together to form a ring and together contain —CH₂—(CH₂)ₙ—CH₂— where n is 0 (i.e., a chemical bond), 1, or 2;
R₄ is hydrogen;
R₅ is hydrogen;
R₆ is hydrogen;
R₇ is hydrogen or alkyl;
R₈ is hydrogen;
R₉ is hydrogen, alkyl, substituted alkyl or COOR^{A}, where R_{A} is alkyl, substituted alkyl, or a pharmaceutically acceptable salt, a prodrug, or a tautomer thereof.

In one embodiment, R₁ is hydrogen or alkyl and R₂ and R₃ taken together to form a ring and together contain —CH₂—(CH₂)ₙ—CH₂— where n=1 or 2. In another embodiment, R₂ or R₃, or both, are a C₁-C₆ alkyl. For example, either R₂ or R₃, or both, can be ethyl. In another example, R₂ or R₃, or both, are methyl. In another embodiment, R₉ is a C₁-C₆ alkyl. For example, R₉ can be methyl. In still another embodiment, R₉ is COOR^{A}. In one example, R^{A} is tert-butyl. However, the invention is not so limited.

In one embodiment, where R₁ and/or R₉ are substituted alkyl, the alkyl is substituted with a halogen, nitrile or benzene ring. In another embodiment, where R₁ is a cycloalkyl, it is selected from a C₃-C₆ cycloalkyl.

In one embodiment, the invention provides compositions containing compounds of the invention, when provided at a low dose function as progesterone receptor antagonists, and thus, avoid the side effects of agonists which include stimulation of breast and ovary tissue.

In another embodiment, the compound of invention comprises the structure (I), where R₉ is a substituted or unsubstituted C₁-C₆ alkyl, substituted or unsubstituted C₁-C₄ alkyl, or methyl. The inventors have found that compounds of this formula have particularly desirable antagonistic activity. For instance, the 1-alkylpyrrole derivatives listed as the 2^{nd}, 4^{th} and 6^{th} compounds in the TABLE below each exhibit greater potency than the corresponding 1-unsubstituted pyrrole derivative listed as the 1st, 3^{th} and 5^{th} compounds respectively in the TABLE.

In one embodiment, R₁ is hydrogen or C₁-C₆ alkyl, hydrogen or C₁-C₄ alkyl, or hydrogen. R₂ and R₃ are independently selected from hydrogen and C₁-C₆ alkyl, hydrogen and C₁-C₄ alkyl, or hydrogen, methyl and ethyl. Alternatively, R₂ and R₃ represent —CH₂—(CH₂)n-CH₂— where n=1 or 2. R₄ is hydrogen. R₅ is hydrogen. R₆ is hydrogen. R₇ is hydrogen or alkyl, hydrogen or C₁-C₆ alkyl, hydrogen or C₁-C₄ alkyl, or hydrogen. R₈ is hydrogen. R₉ is C₁-C₆ alkyl, C₁-C₄ alkyl, or methyl.

The compounds utilized according to the present invention can contain one or more asymmetric centers and can thus give rise to optical isomers and diastereomers. While shown without respect to stereochemistry, the compounds can include optical isomers and diastereomers; racemic and resolved enantiomerically pure R and S stereoisomers; other mixtures of the R and S stereoisomers; and pharmaceutically acceptable salts thereof.

The term “alkyl” is used herein to refer to both straight- and branched-chain saturated aliphatic hydrocarbon groups having about 1 to about 8 carbon atoms, and preferably about 1 to about 6 carbon atoms (i.e., C₁, C₂, C₃, C₄, C₅ or C₆).

The term “alkenyl” is used herein to refer to both straight- and branched-chain alkyl groups having one or more carbon-carbon double bonds and containing about 3 to about 8 carbon atoms. Preferably, the term alkenyl refers to an alkyl group having 1 or 2 carbon-carbon double bonds and having 3 to about 6 carbon atoms. The term “alkynyl” group is used herein to refer to both straight- and branched-chain alkyl groups having one or more carbon-carbon triple bonds and having 3 to about 8 carbon atoms. Preferably, the term alkynyl refers to an alkyl group having 1 or 2 carbon-carbon triple bonds and having 3 to about 6 carbon atoms.

The terms “substituted alkyl”, “substituted alkenyl”, and “substituted alkynyl” refer to alkyl, alkenyl, and alkynyl groups, respectively, having one or more substituents including, without limitation, halogen, CN, OH, NO₂, amino, aryl, heterocyclic groups, aryl, alkoxy, aryloxy, alkyloxy, alkylcarbonyl, alkylcarboxy, amino, and arylthio which groups can be optionally substituted.

The term “acyl” as used herein refers to a carbonyl substituent, i.e., a C(O)(R) group where R is a straight- or branched-chain saturated aliphatic hydrocarbon group including, without limitation, alkyl, alkenyl, and alkynyl groups. Preferably, the R groups have 1 to about 8 carbon atoms, and more preferably 1 to about 6 carbon atoms. The term “substituted acyl” refers to an acyl group which is substituted with 1 or more groups including halogen, CN, OH, and NO₂.

The term “aryl” as used herein refers to an aromatic system which can include a single ring or multiple aromatic rings fused or linked together where at least one part of the fused or linked rings forms the conjugated aromatic system. The aryl groups include, but are not limited to, phenyl, naphthyl, biphenyl, anthryl, tetrahydronaphthyl, phenanthryl, indene, benzonaphthyl, fluorenyl, and carbazolyl. The term “substituted aryl” refers to an aryl group which is substituted with one or more substituents including halogen, CN, OH, NO₂, amino, alkyl, cycloalkyl, alkenyl, alkynyl, alkoxy, aryloxy, alkyloxy, alkylcarbonyl, alkylcarboxy, alkylamino, and arylthio, which groups can be optionally substituted. Preferably, a substituted aryl group is substituted with 1 to about 4 substituents.

The term “heterocyclic” as used herein refers to a stable 4- to 7-membered monocyclic or multicyclic heterocyclic ring which is saturated, partially unsaturated, or wholly unsaturated. The heterocyclic ring has in its backbone carbon atoms and one or more heteroatoms including nitrogen, oxygen, and sulfur atoms. Preferably, the heterocyclic ring has about 1 to about 4 heteroatoms in the backbone of the ring. When the heterocyclic ring contains nitrogen or sulfur atoms in the backbone of the ring, the nitrogen or sulfur atoms can be oxidized. The term “heterocyclic” also refers to multicyclic rings in which a heterocyclic ring is fused to an aryl ring. The heterocyclic ring can be attached to the aryl ring through a heteroatom or carbon atom provided the resultant heterocyclic ring structure is chemically stable.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Oxygen-containing rings include, but are not limited to, furyl, tetrahydrofuranyl, pyranyl, pyronyl, and dioxinyl rings. Nitrogen-containing rings include, without limitation, pyrrolyl, pyrazolyl, imidazolyl, triazolyl, pyridyl, piperidinyl, 2-oxopiperidinyl, pyridazinyl, pyrimidinyl, pyrazinyl, piperazinyl, azepinyl, triazinyl, pyrrolidinyl, and azepinyl rings. Sulfur-containing rings include, without limitation, thienyl and dithiolyl rings. Mixed heteroatom containing rings include, but are not limited to, oxathiolyl, oxazolyl, thiazolyl, oxadiazolyl, oxatriazolyl, dioxazolyl, oxathiazolyl, oxathiolyl, oxazinyl, oxathiazinyl, morpholinyl, thiamorpholinyl, thiamorpholinyl sulfoxide, oxepinyl, thiepinyl, and diazepinyl rings. Fused heteroatom-containing rings include, but are not limited to, benzofuranyl, thionapthene, indolyl, benazazolyl, purindinyl, pyranopyrrolyl, isoindazolyl, indoxazinyl, benzoxazolyl, anthranilyl, benzopyranyl, quinolinyl, isoquinolinyl, benzodiazonyl, napthylridinyl, benzothienyl, pyridopyridinyl, benzoxazinyl, xanthenyl, acridinyl, and purinyl rings.

The term “substituted heterocyclic” as used herein refers to a heterocyclic group having one or more substituents including halogen, CN, OH, NO₂, amino, alkyl, cycloalkyl, alkenyl, alkynyl, alkoxy, aryloxy, alkyloxy, alkylcarbonyl, alkylcarboxy, alkylamino, and arylthio, which groups can be optionally substituted. Preferably, a substituted heterocyclic group has 1 to 4 substituents.

The term “arylthio” as used herein refers to the S(aryl) group, where the point of attachment is through the sulfur-atom and the aryl group can be optionally substituted. The term “alkoxy” as used herein refers to the O(alkyl) group, where the point of attachment is through the oxygen-atom and the alkyl group is optionally substituted. The term “aryloxy” as used herein refers to the O(aryl) group, where the point of attachment is through the oxygen-atom and the aryl group is optionally substituted.

The term “alkylcarbonyl” as used herein refers to the C(O)(alkyl) group, where the point of attachment is through the carbon-atom of the carbonyl moiety and the alkyl group is optionally substituted.

The term “alkylcarboxy” as used herein refers to the C(O)O(alkyl) group, where the point of attachment is through the carbon-atom of the carboxy moiety and the alkyl group is optionally substituted.

The term “aminoalkyl” as used herein refers to both secondary and tertiary amines where the point of attachment is through the nitrogen-atom and the alkyl groups are optionally substituted. The alkyl groups can be the same or different.

The term “halogen” as used herein refers to Cl, Br, F, or I groups.

The compounds of the present invention encompass tautomeric forms of the structures provided herein characterized by the bioactivity of the drawn structures. Further, the compounds of the present invention can be used in the form of salts derived from pharmaceutically or physiologically acceptable acids, bases, alkali metals and alkaline earth metals.

Pharmaceutically acceptable salts can be formed from organic and inorganic acids, for example, acetic, propionic, lactic, citric, tartaric, succinic, fumaric, maleic, malonic, mandelic, malic, phthalic, hydrochloric, hydrobromic, phosphoric, nitric, sulfuric, methanesulfonic, napthalenesulfonic, benzenesulfonic, toluenesulfonic, camphorsulfonic, and similarly known acceptable acids. Salts may also be formed from inorganic bases, preferably alkali metal salts, for example, sodium, lithium, or potassium, and organic bases, such as ammonium, mono-, di-, and trimethylammonium, mono-, di- and triethylammonium, mono-, di- and tripropylammonium (iso and normal), ethyldimethylammonium, benzyldimethylammonium, cyclohexylammonium, benzylammonium, dibenzylammonium, piperidinium, morpholinium, pyrrolidinium, piperazinium, 1-methylpiperidinium, 4-ethylmorpholinium, 1-isopropylpyrrolidinium, 1,4-dimethylpiperazinium, 1-n-butyl piperidinium, 2-methylpiperidinium, 1-ethyl-2-methylpiperidinium, mono-, di- and triethanolammonium, ethyl diethanolammonium, n-butylmonoethanolammonium, tris(hydroxymethyl)methylammonium, phenylmonoethanolammonium, and the like.

Physiologically acceptable alkali salts and alkaline earth metal salts can include, without limitation, sodium, potassium, calcium and magnesium salts in the form of esters, and carbamates. Other conventional “pro-drug” forms can also be utilized which, when delivered in such form, convert to the active moiety in vivo.

These salts, as well as other compounds of the invention can be in the form of esters, carbamates and other conventional “pro-drug” forms, which, when administered in such form, convert to the active moiety in vivo. In a currently preferred embodiment, the prodrugs are esters. See, e.g., B. Testa and J. Caldwell, “Prodrugs Revisited: The “Ad Hoc” Approach as a Complement to Ligand Design”, Medicinal Research Reviews, 16(3):233-241, ed., John Wiley & Sons (1996).

As described herein, the compounds of formula I and/or salts, prodrugs or tautomers thereof, are delivered in contraceptive or other therapeutic/prophylactic regimens.

The compounds discussed herein also encompass “metabolites” which are unique products formed by processing the compounds of the invention by the cell or patient. Preferably, metabolites are formed in vivo.

The compounds of this invention are readily prepared by one of skill in the art according to the following schemes from commercially available starting materials or starting materials which can be prepared using literature procedures. These schemes show the preparation of representative compounds of this invention. Variations on these methods, or other methods known in the art can be readily utilized by one of skill in the art given the information provided herein.

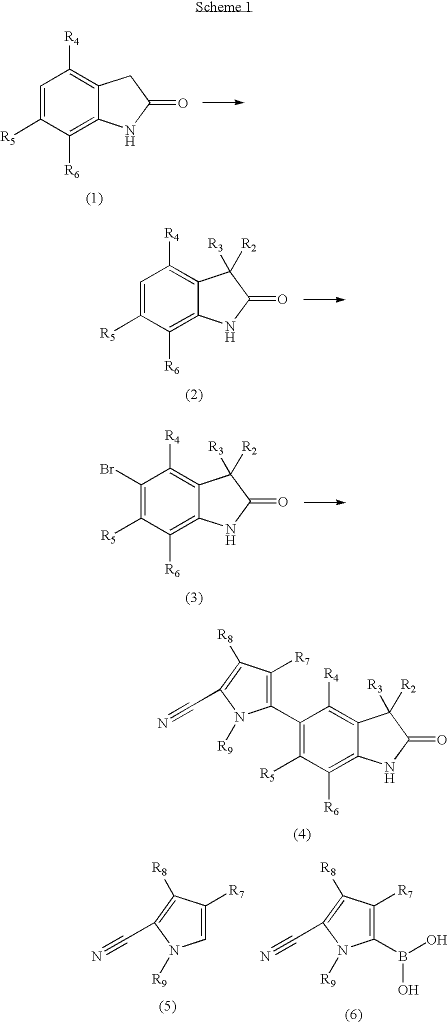

According to scheme 1, an appropriately substituted oxindole (1) is treated with a suitable base (normally 2 or more molar equivalents) and an alkylating agent to afford substituted oxindoles (2). The range of suitable bases includes alkyl lithium bases, potassium tertiary butoxide, sodium hexamethyldisilazide and similar bases. The base may also be used in conjunction with an additive. Generally the compounds of the invention were prepared using n-butyl lithium as the base in anhydrous THF in the presence of lithium chloride or copper bromide. The alkylating agent is normally an alkyl halide (e.g., bromide or iodide) but could also be a triflate, tosylate or mesylate. If one equivalent of alkylating agent is used then the resultant oxindole will be mono-substituted. With two equivalents, then the oxindole will be di-substituted. If the alkylating agent is bifunctional (e.g., a halide or other leaving group at both ends of an alkyl chain) then a spirocyclic ring is produced.

Oxindoles (2) are then brominated to give compound (3). The bromination is conveniently carried out with bromine in a solvent such as methylene chloride or acetic acid, which may be buffered with an additive such as sodium acetate. The bromination may also be accomplished with N-bromosucinimide or pyridinium bromide per bromide. Compound (3) is then converted into compound (4) under the action of a palladium catalyst and a suitable coupling partner. The coupling partner may be formed in situ from the pyrrole (5) and lithium di-isopropylamide and a trialkyl borate or may be the pre-formed boronic acid (6). The source of palladium is normally tetrakis(triphenylphosphine) palladium (0) or another suitable source such as palladium dibenzylidene acetone in the presence of tributylphosphine (tri-tert-butyl phosphine)(Fu, G. C. et al. Journal of the American Chemical Society, 2000, 122, 4020; for alternate catalyst systems see also Hartwig, J. F. et al. Journal of Organic Chemistry, 2002, 67, 5553). A base is also required in the reaction; the normal choices are sodium or potassium carbonate, cesium fluoride, potassium fluoride, potassium phosphate or a tertiary amine base such as triethylamine. The choice of solvents includes THF, dimethoxy ethane, dioxane, ethanol, water, and toluene amongst others. Depending on the reactivity of the coupling partners and reagents, the reaction may be conducted up to the boiling point of the solvents, or may indeed be accelerated under microwave irradiation, if necessary.

Alternatively, compounds (1) to (3) can be prepared according to the routes described in U.S. Provisional Patent Application Nos. 60/676,149 and 60/676,381 (both filed on April 29, 2005), which are hereby incorporated by reference in their entirety.

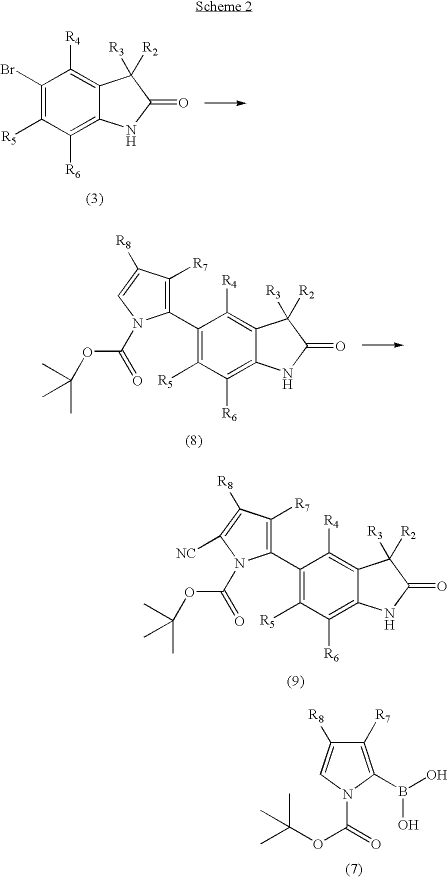

An alternative strategy may be used when R₉=hydrogen, scheme 2. Thus the bromide (3) is coupled with a pyrolle boronic acid of formula (7) under conditions as described above. Compound (8) may then be converted into the nitrile (9). This is most conveniently accomplished by the action of chlorosulfonylisocyanate followed by treatment with DMF, although other methods are also available. The t-butylcarbonate protecting group is then removed to afford the product (4), R₉=H.

When R₁ is to be a substituted alkyl group, then compound (4) is treated with a suitable base (for example sodium hydride, potassium tert-butoxide or cesium carbonate) in a solvent such as THF or DMF, followed by treatment with the appropriate alkylating agent. The alkylating agent would normally be an alkyl halide, or an alkyl sulfonate (tosylate, mesylate or triflate for example).

This invention includes pharmaceutical compositions comprising one or more compounds of this invention and a pharmaceutically acceptable carrier or excipient. The invention also includes methods of treatment which comprise administering to a mammal a pharmaceutically effective amount of one or more compounds as described above as antagonists of the progesterone receptor.

The compounds of this invention can be utilized in methods of contraception, hormone replacement therapy, and the treatment and/or prevention of benign and malignant neoplastic disease. Specific uses of the compounds and pharmaceutical compositions of invention include the treatment and/or prevention of uterine myometrial fibroids, endometriosis, benign prostatic hypertrophy; carcinomas and adenocarcinomas of the endometrium, ovary, breast, colon, prostate, pituitary, meningioma and other hormone-dependent tumors. Additional uses of the present progesterone receptor antagonists include the synchronization of the estrus in livestock, treatment of dysmenorrhea, treatment of dysfunctional uterine bleeding, induction of amenorrhea, and treatment of the symptoms of premenstrual syndrome and premenstrual dysphoric disorder.

In one embodiment, the invention provides compositions containing compounds of the invention, when provided at a low dose function as progesterone receptor antagonists, and thus, avoid the side effects of agonists which include stimulation of breast and ovary tissue.

The effective dosage of active ingredient employed may vary depending on the particular compound employed, the mode of administration and the severity of the condition being treated. However, in general, satisfactory results are obtained when the compounds of the invention are administered at a daily dosage of from about 0.2 mg to about 100 mg, or given in divided doses one to four times a day, or in a sustained release form. Such sustained release formulations are known to those of skill in the art. For most large mammals, the total daily dosage is from about 0.2 mg to 100 mg, from about 0.5 to 80 mg, or about 1 mg to 50 mg. This dosage regimen may be adjusted to provide the optimal therapeutic response. For example, several divided doses may be administered daily or the dose may be proportionally reduced as indicated by the exigencies of the therapeutic situation.

When the compounds are employed for the above utilities, they may be combined with one or more pharmaceutically acceptable carriers or excipients, for example, solvents, diluents and the like, and may be administered orally in such forms as tablets, capsules, dispersible powders, granules, or suspensions containing, for example, from about 0.05 to 5% of suspending agent, syrups containing, for example, from about 10 to 50% of sugar, and elixirs containing, for example, from about 20 to 50% ethanol, and the like, or parenterally in the form of sterile injectable solutions or suspensions containing from about 0.05 to 5% suspending agent in an isotonic medium. Such pharmaceutical preparations may contain, for example, from about 25 to about 90% of the active ingredient in combination with the carrier, more usually between about 5% and 60% by weight.

These active compounds may be administered orally as well as by intravenous, intramuscular, or subcutaneous routes. Solid carriers include starch, lactose, dicalcium phosphate, microcrystalline cellulose, sucrose and kaolin, while liquid carriers include sterile water, polyethylene glycols, non-ionic surfactants and edible oils such as corn, peanut and sesame oils, as are appropriate to the nature of the active ingredient and the particular form of administration desired. Adjuvants customarily employed in the preparation of pharmaceutical compositions may be advantageously included, such as flavoring agents, coloring agents, preserving agents, and antioxidants, for example, vitamin E, ascorbic acid, BHT and BHA.

The preferred pharmaceutical compositions from the standpoint of ease of preparation and administration are solid compositions, particularly tablets and hard-filled or liquid-filled capsules. Oral administration of the compounds is preferred. These active compounds may also be administered parenterally or intraperitoneally. Solutions or suspensions of these active compounds as a free base or pharmacologically acceptable salt can be prepared in water suitably mixed with a surfactant such as hydroxypropylcellulose. Dispersions can also be prepared in glycerol, liquid, polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringe ability exits. It must be stable under conditions of manufacture and storage and must be preserved against the contaminating action of microorganisms such as bacterial and fungi. The carrier can be a solvent or dispersion medium containing, for example, water, ethanol (e.g., glycerol, propylene glycol and liquid polyethylene glycol), suitable mixtures thereof, and vegetable oil.

In this disclosure, the terms anti-progestational agents, anti-progestins, and progesterone receptor antagonists (PR antagonists) are understood to be synonymous. Similarly, progestins, progestational agents and progesterone receptor agonists (PR agonists) are understood to refer to compounds of the same activity.

The use of this invention includes cyclic regimens involving administration of a PR antagonist of the invention alone. In another embodiment, the cyclic regimen involves administration of a PR antagonist of the invention in combination with an estrogen or progestin or both. Particularly desirable progestins can be selected from among those described in U.S. Pat. No. 6,355,648; U.S. Pat. No. 6,521,657; U.S. Pat. No. 6,436,929; U.S. Pat. No. 6,540,710; U.S. Pat. No. 6,562,857; and U.S. patent Publication No. 2004-0006060-A1. Still other progestins are known in the art and can be readily selected. In one embodiment, combination regimens include the PR agonist (i.e., progestin) tanaproget [5-(4,4-dimethyl-2-thioxo-1,4-dihydro-2H-3,1-benzoxazin-6-yl)-1-methyl-1H-pyrrole-2-carbonitrile].

This invention further includes administration regimens carried out over 28 consecutive days. These regimens may be continuous, or may involve a terminal portion of the cycle, e.g., 0 to 7 days, containing administration of no progestins, estrogens or anti-progestins.

The regimens described herein may be utilized for contraception, or for any of the other indications described herein. Where administration is for contraception, the compositions may be formulated in oral dosage units.

When utilized for contraception, the PR antagonists of the invention may be administered to a female of child bearing age, alone or in combination with an estrogen. For the first 14-24 days of the cycle, progestins may be administered at a dosage range equal in progestational activity to about 35 μg to about 150 μg levonorgestrel per day, preferably equal in activity to from about 35 μg to about 100 μg levonorgestrel per day. A PR antagonist may then be administered alone or in combination with an estrogen for a period of 1 to 11 days to begin on any cycle day between day 14 and 24. The PR antagonist in these combinations may be administered at a dose of from about 2 μg to about 50 μg per day and the estrogen may be administered at a dose of from about 10 μg to about 35 μg per day. In an oral administration, a package or kit containing 28 tablets may include a placebo tablet on those days when the PR antagonist of the invention or progestin or estrogen is not administered.

In a preferred embodiment of this invention, the compounds of this invention may be administered alone or in combination with estrogen for the initial 18 to 21 days of a 28-day cycle, followed by administration of a compound of the invention, alone or in combination with an estrogen, for from 1 to 7 days. The estrogen to be used in the combinations and formulations of this invention is preferably ethinyl estradiol.

Progestational agents useful with this invention include, but are not limited to, levonorgestrel, norgestrel, desogestrel, 3-ketodesogestrel, norethindrone, gestodene, norethindrone acetate, norgestimate, osaterone, cyproterone acetate, trimegestone, dienogest, drospirenone, nomegestrol, or (17-deacetyl)norgestimate. Among the preferred progestins for use in the combinations of this invention are levonorgestrel, gestodene, trimegestone, and tanaproget.

Examples of orally administered regimens of this invention over a 28 day cycle include administration of progestational agent solely for the first 21 days at a daily dose equal in progestational activity to from about 35 to about 100 μg of levonorgestrel. A PR antagonist compound of this invention can then be administered at a daily dose of from about 2 to 50 mg from day 22 to day 24, followed by no administration or administration of a placebo for days 25 to 28. It is most preferred that the daily dosages of each relevant active ingredient be incorporated into a combined, single daily dosage unit, totaling 28 daily units per 28-day cycle.

In another regimen, a progestational agent may be coadministered for the first 21 days at a daily dose equal in progestational activity to from about 35 to about 150 μg levonorgestrel, preferably equal in activity to from about 35 to about 100 μg levonorgestrel, with an estrogen, such as ethinyl estradiol, at a daily dose range of from about 10 to about 35 μg. This may be followed as described above with a PR antagonist of the invention administered at a daily dose of from about 2 to 50 mg from day 22 to day 24, followed by no administration or administration of a placebo for days 25 to 28.

Still another regimen within the scope of this invention will include coadministration from days 1 to 21 of a progestational agent, the progestational agent, preferably levonorgestrel, being administered at a daily dose equal in progestational activity to from about 35 to about 100 μg levonorgestrel, and an estrogen, such as ethinyl estradiol, at a daily dose range of from about 10 to about 35 μg. This will be followed on days 22 to 24 by coadministration of a PR antagonist of the invention (2 to 50 mg/day) and an estrogen, such as ethinyl estradiol, at a daily dose of from about 10 to about 35 μg. From day 25 to day 28, this regimen may be followed by no administration or administration of a placebo.

This invention also includes kits or packages of pharmaceutical formulations designed for use in the regimens described herein. These kits are preferably designed for daily oral administration over a 28-day cycle, preferably for one oral administration per day, and organized so as to indicate a single oral formulation or combination of oral formulations to be taken on each day of the 28-day cycle. Preferably each kit will include oral tablets to be taken on each the days specified, preferably one oral tablet will contain each of the combined daily dosages indicated.

According to the regimens described above, one 28-day kit may comprise:
a) an initial phase of from 14 to 21 daily dosage units of a progestational agent equal in progestational activity to about 35 to about 150 μg levonorgestrel, preferably equal in progestational activity to about 35 to about 100 μg levonorgestrel;
b) a second phase of from 1 to 11 daily dosage units of a PR antagonist compound of this invention, each daily dosage unit containing an antiprogestin compound at a daily dosage of from about 2 to 50 mg; and
c) optionally, a third phase of an orally and pharmaceutically acceptable placebo for the remaining days of the cycle in which no antiprogestin, progestin or estrogen is administered.

In one embodiment of this kit, the initial phase involves 21 daily dosage units as described in the preceding passage, a second phase of 3 daily dosage units for days 22 to 24 of a PR antagonist compound of this invention and an optional third phase of 4 daily units of an orally and pharmaceutically acceptable placebo for each of days 25 to 28.

In another embodiment, a 28-day cycle packaging regimen or kit of this invention contains, a first phase of from 18 to 21 daily dosage units, and more desirably, 21 days, as described in the preceding passages, and further includes, as an estrogen, ethinyl estradiol, at a daily dose range of from about 10 to about 35 μg; b) a second phase of from 1 to 7 daily dosage units, and preferably, 4 daily dosage units, as described above, and an optional placebo for each of the remaining 0-9 days, or about 4 days, in the 28-day cycle in which no progestational agent, estrogen or antiprogestin is administered.

A further 28-day packaged regimen or kit of this invention comprises:
a) a first phase of from 18 to 21 daily dosage units, each containing a progestational agent of this invention at a daily dose equal in progestational activity to about 35 to about 150 μg levonorgestrel, preferably equal in activity to from about 35 to about 100 μg levonorgestrel, and ethinyl estradiol at a daily dose range of from about 10 to about 35 μg;
b) a second phase of from 1 to 7 daily dose units, each daily dose unit containing an antiprogestin of this invention at a concentration of from 2 to 50 mg; and ethinyl estradiol at a concentration of from about 10 to about 35 μg; and
c) optionally, an orally and pharmaceutically acceptable placebo for each of the remaining 0-9 days in the 28-day cycle in which no progestational agent, estrogen or antiprogestin is administered.

In one embodiment, the package or kit just described comprises a first phase of 21 daily dosage units; a second phase of 3 daily dose units for days 22 to 24, each dose unit containing an antiprogestin of this invention at a concentration of from 2 to 50 mg; and ethinyl estradiol at a concentration of from about 10 to about 35 μg; and optionally, a third phase of 4 daily units of an orally and pharmaceutically acceptable placebo for each of days 25 to 28.

In each of the regimens, kits, and packages just described, it is preferred that the daily dosage of each pharmaceutically active component of the regimen remain fixed in each particular phase in which it is administered. It is also understood that the daily dose units described are to be administered in the order described, with the first phase followed in order by the second and third phases. To help facilitate compliance with each regimen, it is also preferred that the kits contain the placebo described for the final days of the cycle. It is further preferred that each package or kit comprise a pharmaceutically acceptable package having indicators for each day of the 28-day cycle, such as a labeled blister package or dial dispenser package known in the art.

These dosage regimens may be adjusted to provide the optimal therapeutic response. For example, several divided doses of each component may be administered daily or the dose may be proportionally increased or reduced as indicated by the exigencies of the therapeutic situation. In the descriptions herein, reference to a daily dosage unit may also include divided units which are administered over the course of each day of the cycle contemplated.

The preferred pharmaceutical compositions from the standpoint of ease of preparation and administration are solid compositions, particularly tablets and hard-filled or liquid-filled capsules. Oral administration of the compounds is preferred.

These active compounds may also be administered via a vaginal ring. Suitably, use of the vaginal ring is timed to the 28 day cycle. In one embodiment, the ring is inserted into the vagina, and it remains in place for 3 weeks. During the fourth week, the vaginal ring is removed and menses occurs. The following week a new ring is inserted to be a new regimen. In another embodiment, the vaginal ring is inserted weekly, and is replaced for three consecutive weeks. Then, following one week without the ring, a new ring is inserted to begin a new regimen. In yet another embodiment, the vaginal ring is inserted for longer, or shorter periods of time.

For use in the vaginal ring, a PR antagonist compound is formulated in a manner similar to that described for contraceptive compounds previously described for delivery via a vaginal ring. See, e.g., U.S. Pat. Nos. 5,972,372; 6,126,958; and 6,125,850.

In still another aspect of the invention, the PR antagonist compound(s) are delivered via a transdermal patch. Suitably, use of the patch is timed to the 28 day cycle. In one embodiment, the patch is applied via a suitable adhesive on the skin, where it remains in place for 1 week and is replaced weekly for a total period of three weeks. During the fourth week, no patch is applied and menses occurs. The following week a new patch is applied to be worn to begin a new regimen. In yet another embodiment, the patch remains in place for longer, or shorter periods of time.

The invention further provides kits and delivery devices containing the compounds of the invention for a variety of other therapeutic uses as described herein including, e.g., hormone replacement therapy, the treatment and/or prevention of benign and malignant neoplastic disease. Such kits contain components in addition to the compounds of the invention, including, e.g., instructions for delivery of the compounds of the invention, diluents, vials, syringes, packaging, among other items.

Such kits may optionally be adapted for the selected application, e.g., hormone replacement therapy, treatment and/or prevention of uterine myometrial fibroids, endometriosis, benign prostatic hypertrophy; carcinomas and adenocarcinomas of the endometrium, ovary, breast, colon, prostate, pituitary, meningioma and other hormone-dependent tumors, or the synchronization of the estrus in livestock.

The following examples are provided to illustrate the invention and do not limit the scope thereof. One skilled in the art will appreciate that although specific reagents and conditions are outlined in the following examples, modifications can be made which are meant to be encompassed by the spirit and scope of the invention.

### EXAMPLE 1

### 5-(3,3-dimethyl-2-oxo-2,3-dihydro-1H-indol-5-yl)-1-methyl-1H-pyrrole-2-carbonitrile

A solution of 1-methyl-1H-pyrrole-2-carbonitrile (2.3 g, 21.5 mmol) in anhydrous THF (20 ml) was cooled to 0° C. Tri-iso-propyl borate (5.0 ml, 21.5 mmol) was added followed by drop-wise addition of lithium di-iso-propylamide (14 ml, 2 M solution in heptane/THF/ethylbenzene, 28 mmol). After stirring for 1 hr, water (10 ml) was added followed by sodium carbonate (4.5 g, 43 mmol) and 5-bromo-3,3-dimethyl-1,3-dihydro-indol-2-one (2.40 g, 10 mmol, CAS 120902-45-6, prepared according to International Patent Publication No. WO 00/66556). The mixture was degassed by a stream of nitrogen gas, then tetrakis-(triphenylphosphine)palladium 0 (0.25 g) was added and the mixture heated to reflux under a nitrogen atmosphere. After 16 hours, the mixture was cooled and partitioned between water and ethyl acetate. The aqueous layer was re-extracted with ethylacetate, then the combined organic layers were washed with water, dried (anhyd. MgSO₄) and evaporated. The residue was purified by silica gel column chromatography (hexane: ethylacetate, 5:1 to 3:2) to afford 5-(3,3-dimethyl-2-oxo-2,3-dihydro-1H-indol-5-yl)-1-methyl-1H-pyrrole-2-carbonitrile as a white powder (0.131 g, 0.49 mmol, 5%): HRMS: calc'd for C₁₆H₁₅N₃O, 265.1215; found (ESI, [M+H]⁺), 266.1298; MS (ESI) m/z 266; MS (ESI) m/z 264.

Analytical HPLC: No impurities detected at 210-370 nm window. No impurities detected at 290 nm (max. abs), the Xterra RP18 column, 3.5 μ, 150×4.6 mm, 85/15-5/95 (Ammon. Form. Buff. pH=3.5/ACN+MeOH) for 10 min, hold 4 min, 1.2 mL/min, 5 μl.

### EXAMPLE 2

### 1-methyl-5-(2′-oxo-1′,2′-dihydrospiro[cyclobutane-1,3′-indol]-5′-yl)-1H-pyrrole-2-carbonitrile

A solution of 1-methyl-1H-pyrrole-2-carbonitrile (1.25 g, 11.87 mmol) in anhydrous THF (20 ml) was cooled to 0° C. Tri-iso-propyl borate (2.73 ml, 11.8 mmol) was added followed by dropwise addition of lithium di-iso-propylamide (7.6 ml, 2 M solution in heptane/THF/ethylbenzene, 15.2 mmol). After stirring for 1 hr, water (10 ml) was added followed by potassium carbonate (3.27 g, 23.7 mmol) and 5′-bromospiro[cyclobutane-1,3′-indol]-2′(1′H)-one (1.38 g, 5.49 mmol, CAS 304876-39-9, prepared according to International Patent Publication No. WO 00/66556). The mixture was degassed by a stream of nitrogen gas, then tetrakis(triphenylphosphine) palladium 0 (0.30 g) was added and the mixture heated to reflux under a nitrogen atmosphere. After 16 hrs., the mixture was cooled and partitioned between water and ethyl acetate. The aqueous layer was re-extracted with ethylacetate, then the combined organic layers were washed with water, dried (anhyd. MgSO₄) and evaporated. The residue was purified by silica gel column chromatography (hexane: ethylacetate, gradient elution) to afford 1-methyl-5-(2′-oxo-1′,2′-dihydrospiro[cyclobutane-1,3′-indol]-5′-yl)-1H-pyrrole-2-carbonitrile (0.096 g, 0.34 mmol, 6.2%) as a white powder: MS (ESI) m/z 278; MS (ESI) m/z 276; HRMS: calcd for C₁₇H₁₅N₃O, 277.1215; found (ESI, [M+H]⁺), 278.1295; Major=99.6% at 210-370 nm window; and =99.7% at 290 nm (max. abs) RT=8.9, the Xterra RP18 column, 3.5 μ, 150×4.6 mm, 85/15-5/95 (Ammon. Form. Buff. pH=3.5/ACN+MeOH) for 10 min, hold 4 min.

### EXAMPLE 3

### 5-(3,3-Diethyl-2-oxo-2,3-dihydro-1H-indol-5-yl)-1-methyl-1H-pyrrole-2-carbonitrile

### A. 5-Bromo-3,3-diethyl-1,3-dihydro-indol-2-one

Bromine (0.13 mL, 2.6 mmol) and acetic acid (0.3 ml) were added to a solution of 3,3-diethyl-1,3-dihydro-indol-2-one (0.5 g, 2.6 mmol) and sodium acetate (0.2 g, 2.6 mmol) in dry chloroform (10 ml) at room temperature. After 1 h the reaction was diluted with chloroform and washed with sat. sodium bicarbonate (3×100 mL). The organic layer was dried over anhydrous sodium sulfate, filtered and concentrated in vacuo to give 600 mg (85%) of 5-bromo-3,3-diethyl-1,3-dihydro-indol-2-one (0.60 g, 85%) as a light yellow solid. This compound was used without further purification.

### B. 5-(3,3-Diethyl-2-oxo-2,3-dihydro-1H-indol-5-yl)-1-methyl-1H-pyrrole-2-carbonitrile

1-Methyl-1H-pyrrole-2-carbonitrile (1.2 g; 11.3 mmol) in dry THF (35 ml) was cooled to 0° C. Tri-iso-propyl borate (2.6 mL, 11.3 mmol) was added followed by lithium di-iso-propylamine (7.3 mL, 2.0 M, 14.7 mmol in THF/hexane/ethylbenzene). The dark brown mixture was allowed to warm to room temperature and stirred for 2 h. The reaction was quenched with saturated ammonium chloride (50 mL) and extracted with ethyl acetate (3×100 mL). The organic layer was dried over anhydrous sodium sulfate, filtered and concentrated in vacuo to give the boronic acid.

After evacuation under vacuum and purging with nitrogen, Tetrakis(triphenylphosphine)palladium(0)(0.26 g, 0.2 mmol) was added to a solution of 5-bromo-3,3-diethyl-1,3-dihydro-indol-2-one (0.60 mg, 2.2 mmol) in dry THF (55 mL). After 20 minutes K₂CO₃ (1.5 g, 11.1 mmol) and the above prepared boronic acid were added, followed by water (13 mL). The mixture was heated to 60° C. overnight. The reaction mixture was cooled, filtered through Celite which was rinsed with ethyl acetate. The filtrate was washed with water and brine. The organic layer was dried over anhydrous sodium sulfate, filtered and concentrated in vacuo to give the crude product which was purified by silica gel chromatography methanol:dichloromethane, gradient elution) to give 5-(3,3-diethyl-2-oxo-2,3-dihydro-1H-indol-5-yl)-1-methyl-1H-pyrrole-2-carbonitrile (320 mg, 49%) as a yellow solid. mp 233-235° C. HRMS: calcd for C₁₈H₁₉N₃O, 293.1528; found (ESI, [M+H]⁺), 294.1616 Analytical HPLC: no imp detect. at 210-370 nm window; and no imp detect. at 288 nm (max. abs) RT=7.4, 85/15-5/95 (Ammon. Form. Buff. pH=3.5/ACN+MeOH) for 10 min, hold 4 min, the Xterra RP18 column, 3.5 μ, 150×4.6 mm.

### EXAMPLE 4

### 1-methyl-5-(2-oxo-2,3-dihydro-1H-indol-5-yl)-1H-pyrrole-2-carbonitrile

1-Methyl 1-H pyrrole-2-carbonitrile (0.5 g, 4.8 mmol) and tri-iso-propylborate (1.1 mL, 4.8 mmol) was dissolved in THF (12 mL) at ice bath temperature. Lithium di-iso-propylamide (2.5 mL, 2 M in THF/hexanes/diethylbenzene, 5 mmol) was added slowly over a 10 minute period. After a ½ hour the mixture was allowed to warm to room temperature. In a separate flask, 5-bromoindolin-2-one (0.30 g, 1.42 mmol) and tetrakis(triphenylphoshine)palladium(0) (0.08 g) was dissolved THF (12 mL) and stirred 15 minutes. The above prepared reaction mixture was transferred (via pipet) to this solution, followed by potassium carbonate (0.7 g, 5 mmol) and water (6 mL). The mixture was heated under refluxed (3 hours). After cooling to room temperature, the mixture was then poured into water and extracted with ethylacetate, then the organic layer was dried (MgSO₄) and evaporated. The Flash SiO₂ column with 8/2 then 6/4 Hexane/ethylacetate gave 0.035 g, 11%.

HRMS: calcd for C₁₄H₁₁N₃O, 237.0902; found (ESI, [M+H]⁺), 238.0985. Analytical HPLC: no imp detect. at 210-370 nm window; and no imp detect. at 288 nm (max. abs) RT=7.4, 85/15-5/95 (Ammon. Form. Buff. pH=3.5/ACN+MeOH) for 10 min, hold 4 min, the Xterra RP18 column, 3.5 μ, 150×4.6 mm.

### EXAMPLE 5

### 5-(3-ethyl-2-oxo-2,3-dihydro-1H-indol-5-yl)-1-methyl-1H-pyrrole-2-carbonitrile

### A. Preparation of 3-ethyl-1,3-dihydro-indol-2-one

Oxindole (14.0 g; 0.10 mol) was stirred with 14.0 g(0.22 mol) of Lithium bromide in 450 mL of dry THF at −78° C. 89 mL (0.33 mol; 2.5M in hexanes) of n-Butyllithium over 1 h. The resulting yellow precipitate was stirred for 3 h at −78° C. Iodoethane (18.0 mL, 0.22 mol) in 100 mL of dry THF was added drop-wise and the reaction was allowed to warm to room temperature and stirred overnight. The reaction was quenched with sat. ammonium chloride and concentrated to one-half volume. The orange residue was diluted with ethyl acetate and the layers were separated. The organic layer was dried over anhydrous sodium sulfate, filtered and concentrated in vacuo to give 18.0 g of an orange oil. The crude product was purified by flash chromatography using a stepwise gradient of 10:1 to 6:1 hexane: ethyl acetate to afford 3-ethyl-1,3-dihydro-indol-2-one (2.4 g, 12%).

### B. 5-Bromo-3-ethyl-1,3-dihydro-indol-2-one

Bromine (0.38 mL, 7.4 mmol) in dry dichloromethane (10 mL) was added drop-wise to a solution of 3-ethyl-1,3-dihydro-indol-2-one (1.2 g, 7.4 mmol), sodium acetate (0.61 g; 7.4 mmol) and acetic acid (0.42 mL, 7.4 mmol) in dichloromethane (40 mL) at 0° C. After 3 h at 0° C., the reaction mixture was quenched with 5% aqueous sodium thiosulfate and washed with brine. The organic layer was dried over anhydrous sodium sulfate, filtered and concentrated in vacuo to give 2.0 g of crude product. The crude product was purified by flash chromatography (SiO₂, 8:1 to 3:1 hexane: ethyl acetate gradient elution) to afford of 5-bromo-3-ethyl-1,3-dihydro-indol-2-one (0.8 g, 44%):

### C. 5-(3-ethyl-2-oxo-2,3-dihydro-1H-indol-5-yl)-1-methyl-1H-pyrrole-2-carbonitrile

The compound was prepared using the same procedure as used in the preparation of 5-(3,3-Diethyl-2-oxo-2,3-dihydro-1H-indol-5-yl)-1-methyl-1H-pyrrole-2-carbonitrile, using 800 mg(3.3 mmol) of 5-Bromo-3-ethyl-1,3-dihydro-indol-2-one (0.80 g, 3.3 mmol), (5-cyano-1-methyl-1H-pyrrol-2-yl)boronic acid (1.0 g, 6.6 mmol), tetrakis-(triphenylphosphine)palladium(0) (0.38 g, 0.3 mmol), and 2.3 g(16.6 mmol) of potassium carbonate (2.3 g, 16.6 mmol) in 11 mL of water with 55 mL of THF. The crude product was purified on silica using a stepwise gradient of 6:1 to 2:1 hexane: ethyl acetate to recover 5-(3-ethyl-2-oxo-2,3-dihydro-1H-indol-5-yl)-1-methyl-1H-pyrrole-2-carbonitrile (0.42 g, 59%) as a mixture of enantiomers. MS (ESI) m/z 266, 264.

### EXAMPLE 6

### 5-[(3R)-3-ethyl-2-oxo-2,3-dihydro-1H-indol-5-yl]-1-methyl-1H-pyrrole-2-carbonitrile

This compound was prepared from the chiral separation of racemic 5-(3-ethyl-2-oxo-2,3-dihydro-1H-indol-5-yl)-1-methyl-1H-pyrrole-2-carbonitrile using an AD-H column with SFC-CO₂ with 20% ethanol at a rate of 50 mL/min. at 100 bar at 35° C. to recover (210 mg, 42%) of the enantiomer. mp 144-146° C. α_{D}=−37, c=0.01 in DMSO, arbitrarily assigned as 5-[(3R)-3-ethyl-2-oxo-2,3-dihydro-1H-indol-5-yl]-1-methyl-1H-pyrrole-2-carbonitrile.

### EXAMPLE 7

### 5-[(3 S)-3-ethyl-2-oxo-2,3-dihydro-1H-indol-5-yl]-1-methyl-1H-pyrrole-2-carbonitrile

This compound was isolated using the same chiral preparatory method as 5-[(3R)-3-ethyl-2-oxo-2,3-dihydro-1H-indol-5-yl]-1-methyl-1H-pyrrole-2-carbonitrile (290 mg, 58%) of the enantiomer was recovered. mp 145-147° C.-α_{D}=+27, c=0.01 in DMSO, arbitrarily assigned as 5-[(3S)-3-ethyl-2-oxo-2,3-dihydro-1H-indol-5-yl]-1-methyl-1H-pyrrole-2-carbonitrile.

### EXAMPLE 8

### 1-methyl-5-(2′-oxo-1′,2′-dihydrospiro[cyclopropane-1,3′-indol]-5′-yl)-1H-pyrrole-2-carbonitrile

### A. Spiro[cyclopropane-1,3′-[3H]indol]-2′(1′H)-one

Sodium hydride (9.0 g; 0.2 mol, 60% in mineral oil) was added portion-wise to a solution of oxindole (10.0 g, 75 mmol) in of dry DMF (350 mL). After 15 minutes, the reaction was cooled to 0° C. and 1,4 dibromoethane in 100 mL of dry DMF was added over 15 minutes. The dark brown reaction was allowed to warm to room temperature and stirred overnight. The reaction was diluted with ethyl acetate and water was added. The layers were separated and the organic layer was dried over anhydrous sodium sulfate. The organic layer was filtered, concentrated in vacuo to give 20 g of red oil. The crude product was purified on silica using a stepwise gradient of 10% to 20% ethyl acetate: hexane to afford Spiro[cyclopropane-1,3′-[3H]indol]-2′(1′H)-one (2.3 g, 11%).

Bromine (0.15 mL, 3.0 mmol) was added drop-wise to a mixture of spiro[cyclopropane-1,3′-[3H]indol]-2′(1′H)-one (486 mg, 3.05 mmol), 174 μL (3.0 mmol) acetic acid (0.174 mL, 3.0 mmol) and sodium acetate (250 mg, 3.0 mmol) in dry dichloromethane (120 mL). After 4 h, the reaction was washed with 5% sodium thiosulfalte and brine. The organic layer was dried over anhydrous sodium sulfate, filtered and concentrated in vacuo to give 5′-bromospiro[cyclopropane-1,3′-indol]-2′(1′H)-one (0.75 g, 100%) as a white solid. This compound was used without further purification.

### B. 1-methyl-5-(2′-oxo-1′,2′-dihydrospiro[cyclopropane-1,3′-indol]-5′-yl)-1H-pyrrole-2-carbonitrile

This compound was prepared using the same procedure as described in the preparation of 5-(3-Ethyl-2-oxo-2,3-dihydro-1H-indol-5-yl)-1-methyl-1H-pyrrole-2-carbonitrile.

Tetrakis(triphenylphosphine)palladium(0) (0.37 g, 0.32 mmol) was added to a solution of 5′-bromospiro[cyclopropane-1,3′-indol]-2′(1′H)-one (760 mg, 3.19 mmol) in dry THF (25 mL) and stirred at room temperature for 20 minutes. (5-cyano-1-methyl-1H-pyrrol-2-yl)boronic acid (1.2 g, 8.0 mmol) and 5.5 g(40 mmol) of potassium carbonate (5.5 g, 40 mmol) in water (18 mL) were added and the mixture was stirred at 80° C. overnight. After cooling to room temperature, the crude reaction was diluted with ethyl acetate, washed with water, dried (anhyd. Na₂SO₄) and evaporated. The crude product was purified on silica using a step-wise gradient of 1% to 6% methanol: methylene chloride followed by reverse phase preparatory HPLC to recover 130 mg(15%) of 1-methyl-5-(2′-oxo-1′,2′-dihydrospiro[cyclopropane-1,3′-indol]-5′-yl)-1H-pyrrole-2-carbonitrile. mp 226-229° C. Analytical HPLC: Retention time=8.3 min, purity=100% at 210-300 nm, 85/15-5/95 (Ammon. Form. Buff. pH=3.5/ACN+MeOH) for 10 min, hold 4 min, the Xterra RP18 column, 3.5 μ, 150×4.6 mm.

### EXAMPLE 9

### 5-[(3R)-3-ethyl-3-methyl-2-oxo-2,3-dihydro-1H-indol-5-yl]-1-methyl-1H-pyrrole-2-carbonitrile and 5-[(3S)-3-ethyl-3-methyl-2-oxo-2,3-dihydro-1H-indol-5-yl]-1-methyl-1H-pyrrole-2-carbonitrile

### A. 3-ethyl-3-methyl-1,3-dihydro-2H-indol-2-one

3-Methyloxindole (1.5 g, 10.2 mmol) and lithium chloride (1.26 g, 30 mmol) was dissolved in THF (100 mL). The solution was then cooled to −78° C. and n-butyllithium (4.2 mL, 2.5 M in hexanes, 10.5 mmol) was added slowly over a 15 minute period. Ethyl iodide (4.16 mL, 50 mmol) was added and the mixture was allowed to warm to room temperature. After 24 hours, the mixture was poured into water and extracted with ethyl acetate, dried over magnesium sulfate, and concentrated in vacuo. Flash chromatography (SiO₂, Hexane/ethylacetate 9/1 then 8/2) gave 3-ethyl-3-methyl-1,3-dihydro-2H-indol-2-one (0.750 g, 25%):

HRMS [M+H]⁺176.1076

### B. 5-bromo-3-ethyl-3-methyl-1,3-dihydro-2H-indol-2-one

3-Ethyl-3-methyl-1,3-dihydro-2H-indol-2-one (0.70 g, 4 mmol) was dissolved in DCM (40 mL) and acetic acid (1 mL) at room temperature. Bromine (0.21 mL, 4.1 mmol) was added and the solution allowed to stir 24 hours. The reaction mixture was poured into sodium thiosulfate solution, extracted with diethyl ether, dried over magnesium sulfate, evaporated and the crude product triturated with hexane/ethylacetate 5% to give 5-bromo-3-ethyl-3-methyl-1,3-dihydro-2H-indol-2-one (0.600, 60%): HRMS [M-H]⁻254.0185

### C. 5-[(3S)-3-ethyl-3-methyl-2-oxo-2,3-dihydro-1H-indol-5-yl]-1-methyl-1H-pyrrole-2-carbonitrile and 5-[(3R)-3-ethyl-3-methyl-2-oxo-2,3-dihydro-1H-indol-5-yl]-1-methyl-1H-pyrrole-2-carbonitrile

1-methyl 1-H pyrrole-2-carbonitrile (0.31 mL, 3 mmol) and triisopropylborate (0.69 mL, 3 mmol) was dissolved in THF (12 mL) at ice bath temperature. 2M LDA (1.5 mL, 3 mmol) was added slowly over a 10 minute period. After a ½ hour the mixture was allowed to warm to room temperature. In a separate flask, 5-bromo-3-ethyl-3-methyl-1,3-dihydro-2H-indol-2-one (0.253 g, 1 mmol) and tetrakis(triphenylphoshine)palladium(0) 0.100 g was dissolved THF (5 mL) and stirred 15 minutes. The pyrrole triisopropyl borate solution was transferred (via pipet) to this solution, followed by potassium carbonate (0.414 g, 3 mmol) and water (3 mL). The mixture was refluxed 3 hours. The mixture was then poured into water and extracted with ethylacetate. Flash SiO₂ column with 4/1 then 3/2 Hexane/THF gave the racemic product which was separated by chiral hplc: Chiralpak OD-H, 20 mm×250 mm; mobile phase 85/15-5/95 (Ammonium Formate Buffer. pH=3.5/acetontrile+MeOH) for 10 min, hold 4 min. giving 0.062 g and 0.061 g respectively. HRMS [M+H]⁺=280.1450

The first eluting compound, retention time=3.8 min. was arbitrarily assigned as the R-enantiomer. The second eluting compound, retention time=4.38 min. was arbitrarily assigned as the S-enantomer.

### EXAMPLE 10

### 1-methyl-5-(1,3,3-trimethyl-2-oxo-2,3-dihydro-1H-indol-5-yl)-1H-pyrrole-2-carbonitrile

A solution of 5-(3,3-dimethyl-2-oxo-2,3-dihydro-1H-indol-5-yl)-1-methyl-1H-pyrrole-2-carbonitrile (0.50 g, 1.88 mmol) in dry THF (5 ml) was treated with potassium tert-butoxide (1M in THF, 2.25 ml, 2.25 mmol) at room temperature under a nitrogen atmosphere. After 30 min., iodomethane (0.155 ml, 2.5 mmol) was added and the mixture stirred overnight. The reaction mixture was partitioned between ethylacetate and water, the organic layer washed with brine, dried (MgSO₄) and evaporated. The residue was recrystallized from THF/hexane to afford the title compound (0.37 g, 1.24 mmol, 66%) as a white solid.

HRMS, Analytical HPLC: retention time 9.4 min, 210-370 nm, the Xterra RP18 column, 3.5 μ, 150×4.6 mm 40C 85/15-5/95 (Ammon. Form. Buff. pH=3.5/ACN+MeOH) for 10 min, hold 4 min 1.2 mL/min 5 μL injection.

### EXAMPLE 11

### PHARMACOLOGY

Three types of assays are illustrated herein for use in assessing the activity of the compounds of the invention.

A. Effects of Progestins and Antiprogestins on Alkaline Phosphatase Activity in T47D cells (T47D Alkaline Phosphatase Assay)

The molecules of the present invention are anticipated to be active in the antagonist mode in the T47D alkaline phosphatase assay at concentrations of 3 μM or lower.

1. Reagents:

Culture medium: DMEM:F12 (1:1) (GIBCO, BRL) supplemented with 5% (v/v) charcoal stripped fetal bovine serum (not heat-inactivated), 100 U/ml penicillin, 100 μg/ml streptomycin, and 2 mM GlutaMax (GIBCO, BRL).

Alkaline phosphatase assay buffer: I. 0.1M Tris-HCI, pH 9.8, containing 0.2% Triton X-100, 0.1M Tris-HCI, pH 9.8, containing 4 mM p-nitrophenyl phosphate (Sigma).

2. Cell Culture And Treatment:

Frozen T47D cells are thawed in a 37° C. water bath and diluted to 280,000 cells/ml in culture medium. To each well in a 96-well plate (Falcon, Becton Dickinson Labware), 180 μl of diluted cell suspension is added. Twenty μl of reference or test compounds diluted in the culture medium is then added to each well. When testing for progestin antagonist activity, reference antiprogestins or test compounds are added in the presence of 1 nM progesterone. The cells are incubated at 37° C. in a 5% CO₂ humidified atmosphere for 24 hours. For high throughput screening, one concentration of each compound will be tested at 0.3 μg/ml. Based on an average molecular weight of 300 g/mol for the compounds in the library, the concentration is approximately 1 μM. Subsequently, active compounds will be tested in dose response assays to determine EC₅₀ and IC₅₀.

3. Alkaline Phosphatase Enzyme Assay:

At the end of treatment, the medium is removed from the plate. Fifty μl of assay buffer I is added to each well. The plates are shaken in a titer plate shaker for 15 min. Then 150 μl of assay buffer II is added to each well. Optical density measurements are taken at 5 min intervals for 30 min. at a test wavelength of 405 nM.

4. Analysis of Dose-Response Data.

For reference and test compounds, a dose response curve is generated for dose vs. the rate of enzyme reaction (slope). Square root-transformed data are used for analysis of variance and nonlinear dose response curve fitting for both agonist and antagonist modes. Huber weighting is used to down-weight the effects of outliers. EC₅₀ or IC₅₀ values are calculated from the retransformed values. JMP software (SAS Institute, Inc.) is used for both one-way analysis of variance and non-4 linear dose response analysis in both single dose and dose response studies.

5. Reference Compounds:

Progesterone and trimegestone are reference progestins and RU486 is the reference antiprogestin. All reference compounds are run in full dose response curves and the EC₅₀ and IC₅₀ values are calculated.

6. Comparative Study

For example, from U.S. Pat. No. 6,562,857 B2, 5-(spiro[cyclohexane-1,3′-[3H]indole]-2′-oxo-5′-yl)-1H-pyrrole-1-methyl-2-carbonitrile is a progesterone receptor agonist with an EC₅₀=2.8 nM in the T47D cell alkaline phosphatase assay. In contrast 1-methyl-5-(2′-oxo-1′,2′-dihydrospiro[cyclopentane-1,3′-indol]-5′-yl)-1H-pyrrole-2-carbonitrile is a progesterone receptor antagonist in this same assay with an IC₅₀=30 nM.

7. Results

TABLE
T47D Cell Alkaline Phosphatase Assay

| Chemical Name | IC50 (nM) |
|---|---|
| 5-(3,3-Dimethyl-2-oxo-2,3-dihydro-1H-indol-5-yl)-1H- | 27.2 |
| pyrrole-2-carbonitrile | |
| 5-(3,3-dimethyl-2-oxo-2,3-dihydro-1H-indol-5-yl)-1-methyl- | 10 |
| 1H-pyrrole-2-carbonitrile | |
| 5-(1,2-Dihydro-2-oxospiro[cyclopentane-1,3-[3H]indol]-5-yl)- | 60 |
| 1H-pyrrole-2-carbonitrile | |
| 1-methyl-5-(2′-oxo-1′,2′-dihydrospiro[cyclopentane-1,3′- | 30 |
| indol]-5′-yl)-1H-pyrrole-2-carbonitrile | |
| 5-(1,3,3-trimethyl-2-oxo-2,3-dihydro-1H-indol-5-yl)-1H- | 72.9 |
| pyrrole-2-carbonitrile | |
| 1-methyl-5-(1,3,3-trimethyl-2-oxo-2,3-dihydro-1H-indol- | 13.3 |
| 5-yl)-1H-pyrrole-2-carbonitrile | |
| 1-methyl-5-(2′-oxo-1′,2′-dihydrospiro[cyclobutane-1,3′-indol]- | 1 |
| 5′-yl)-1H-pyrrole-2-carbonitrile | |
| 5-(3,3-diethyl-2-oxo-2,3-dihydro-1H-indol-5-yl)-1-methyl-1H- | 3.3 |
| pyrrole-2-carbonitrile | |
| 1-methyl-5-(2-oxo-2,3-dihydro-1H-indol-5-yl)-1H-pyrrole-2- | 20.4 |
| carbonitrile | |
| 5-(3-ethyl-2-oxo-2,3-dihydro-1H-indol-5-yl)-1-methyl-1H- | 5.7 |
| pyrrole-2-carbonitrile | |
| 1-methyl-5-(2′-oxo-1′,2′-dihydrospiro[cyclopropane-1,3′- | 8.1 |
| indol]-5′-yl)-1H-pyrrole-2-carbonitrile | |
| 5-[(3S)-3-ethyl-3-methyl-2-oxo-2,3-dihydro-1H-indol-5-yl]-1- | 7.4 |
| methyl-1H-pyrrole-2-carbonitrile | |
| 5-[(3R)-3-ethyl-3-methyl-2-oxo-2,3-dihydro-1H-indol-5-yl]-1- | 3.2 |
| methyl-1H-pyrrole-2-carbonitrile | |
| 5-[(3R)-3-ethyl-2-oxo-2,3-dihydro-1H-indol-5-yl]-1-methyl- | 6.1 |
| 1H-pyrrole-2-carbonitrile | |
| 5-[(3S)-3-ethyl-2-oxo-2,3-dihydro-1H-indol-5-yl]-1-methyl- | 13.1 |
| 1H-pyrrole-2-carbonitrile | |
| 2-Cyano-5-(1,2-dihydro-2-oxospiro[cyclopentane-1,3- | 300 |
| [3H]indol]-5-yl)-1H-pyrrole-1-carboxylic acid, tert-butyl ester | |

B. Progestational and Antiprogestational Activity in Mature Ovariectomized Rats (Rat Decidualization Assay)

This assay is used to evaluate the effect of progestins and antiprogestins on rat uterine decidualization and compare the relative potencies of various test compounds.

1. Methods and Reagents

Test compounds are dissolved in 100% ethanol and mixed with corn oil (vehicle). Stock solutions of the test compounds in oil (Mazola™) are then prepared by heating (˜80° C.) the mixture to evaporate ethanol. Test compounds are subsequently diluted with 100% corn oil or 10% ethanol in corn oil prior to the treatment of animals. No difference in decidual response was found when these two vehicles were compared.

2. Animals

Ovariectomized mature female Sprague-Dawley rats (˜60-day old and 230 g) are obtained from Taconic (Taconic Farms, NY) following surgery. Ovariectomy is performed at least 10 days prior to treatment to reduce circulating sex steroids. Animals are housed under 12 hr light/dark cycle and given standard rat chow and water ad libitum.

3. Treatment

Rats are weighed and randomly assigned to groups of 4 or 5 before treatment. Test compounds in 0.2 ml vehicle are administered by subcutaneous injection in the nape of the neck or by gavage using 0.5 ml. The animals are treated once daily for seven days. For testing antiprogestins, animals are given the test compounds and a EC₅₀ dose of progesterone (5.6 mg/kg) during the entire treatment period. One group of animals receiving an EC₅₀ dose of progesterone alone serves as a positive control.

4. Dosing

Doses are prepared based upon mg/kg mean group body weight. In all studies, a control group receiving vehicle is included. Determination of dose response curves is carried out using doses with half log increases (e.g., 0.1, 0.3, 1.0, 3.0 mg/kg).

5. Decidual induction

Approximately 24 hr after the third injection, decidualization is induced in one of the uterine horns of anesthetized rats by scratching the antimesometrial luminal epithelium with a blunt 21 G needle. The contralateral horn is not scratched and serves as an unstimulated control. Approximately 24 hr following the final treatment, rats are sacrificed by CO asphyxiation and body weight measured. Uteri are removed and trimmed of fat. Decidualized (D-horn) and control (C-horn) uterine horns are weighed separately.

6. Analysis of Results

In agonist mode, the increase in weight of the decidualized uterine horn is calculated by D-horn/C-horn and logarithmic transformation is used to maximize normality and homogeneity of variance. The Huber M-estimator is used to down weight the outlying transformed observations for both dose-response curve fitting and one-way analysis of variance (ANOVA). EC₅₀ is calculated from the transformed value. In antagonist mode, a square root transformation on raw responses (D-horn/C-horn) is recommended by using maximum likelihood Box-Cox transformation. The Huber weight is used to down weight the outlying transformed observations for dose-response curve fitting and one-way ANOVA. IC₅₀ is calculated from the retransformed value. JMP software (SAS Institute, Inc.) is used for both one-way ANOVA and non-linear dose-response analyses.

7. Reference Compounds

All progestin or antiprogestin reference compounds were run in full dose-response curves and the EC₅₀ or IC₅₀ for decidual response was calculated.

8. Results

5-(3,3-Dimethyl-2-oxo-2,3-dihydro-1H-indol-5-yl)-1-methyl-1H-pyrrole-2-carbonitrile is a PR antagonist in the alkaline phosphatase assay (IC₅₀=10 nM) and is very potent in the rat decidual assay (ED₅₀ =0.2 mg/k po).

All patents, patent publications, and other publications listed in this specification are incorporated herein by reference. While the invention has been described with reference to a particularly preferred embodiment, it will be appreciated that modifications can be made without departing from the spirit of the invention. Such modifications are intended to fall within the scope of the appended claims.

1. A composition comprising a progesterone receptor antagonist comprising a compound of formula I: 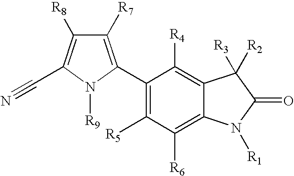
wherein,
R₁ is hydrogen, alkyl, substituted alkyl, cycloalkyl, C₃-C₆ alkenyl, or C₃-C₆ alkynyl;
R₂ and R₃ are each independently selected from: hydrogen, alkyl, substituted alkyl or R₂ and R₃ are taken together to form a ring and together contain —CH₂—(CH₂)n—CH₂— where n is 0, 1, or 2;
R₄ is hydrogen;
R₅ is hydrogen;
R₆ is hydrogen;
R₇ is hydrogen or alkyl;
R₈ is hydrogen;
R₉ is hydrogen, alkyl, substituted alkyl or COOR^{A};
R^{A} is alkyl or substituted alkyl;
or a pharmaceutically acceptable salt thereof.

2. The composition according to claim 1, wherein
R₁ is hydrogen or alkyl;
R₂ and R₃ are taken together to form a ring and together contain —CH₂—(CH₂)ₙ—CH₂—;
n is 1 or 2.

3. The composition according to claim 1, wherein R₂ and R₃ are each an alkyl.

4. The composition according to claim 3, wherein R₂ or R₃ is ethyl.

5. The composition according to claim 3, wherein R₂ or R₃ is methyl.

6. The composition according to claim 1, wherein R₉ is C₁-C₄ alkyl.

7. The composition according to claim 6, wherein R₉ is methyl.

8. The composition according to claim 1, wherein said compound is selected from the group consisting of: 1-methyl-5-(2′-oxo-1′,2′-dihydrospiro[cyclobutane-1,3′-indol]-5′-yl)-1H-pyrrole-2-carbonitrile; 1-methyl-5-(2-oxo-2,3-dihydro-1H-indol-5-yl)-1H-pyrrole-2-carbonitrile; 5-(3-ethyl-2-oxo-2,3-dihydro-1H-indol-5-yl)-1-methyl-1H-pyrrole-2-carbonitrile; 1-methyl-5-(2′-oxo-1′,2′-dihydrospiro[cyclopropane-1,3′-indol]-5′-yl)-1H-pyrrole-2-carbonitrile; 1-methyl-5-(1,3,3-trimethyl-2-oxo-2,3-dihydro-1H-indol-5-yl)-1H-pyrrole-2-carbonitrile

9. The composition according to claim 1, wherein R₉ is COOR^{A} and R^{A} is tert-butyl.

10. The composition according to claim 1, wherein the composition comprises a low dose of a compound selected from the group consisting of: 5-(3,3-dimethyl-2-oxo-2,3-dihydro-1H-indol-5-yl)-1-methyl-1H-pyrrole-2-carbonitrile; 5-[(3R)-3-ethyl-2-oxo-2,3-dihydro-1H-indol-5-yl]-1-methyl-1H-pyrrole-2-carbonitrile; 5-[(3S)-3-ethyl-2-oxo-2,3-dihydro-1H-indol-5-yl]-1-methyl-1H-pyrrole-2-carbonitrile; 5-[(3R)-3-ethyl-3-methyl-2-oxo-2,3-dihydro-1H-indol-5-yl]-1-methyl-1H-pyrrole-2-carbonitrile; and 5-[(3S)-3-ethyl-3-methyl-2-oxo-2,3-dihydro-1H-indol-5-yl]-1-methyl-1H-pyrrole-2-carbonitrile.

11. The composition according to claim 1 further comprising a pharmaceutically acceptable carrier or excipient.

12. A method for inducing contraception in a mammal, the method comprising administering to a mammal in need thereof a pharmaceutically effective amount of a composition of claim 1.

13. A method for hormone replacement therapy in a mammal, the method comprising administering to a mammal in need thereof a pharmaceutically effective amount of a composition of claim 1.

14. A method for treating hormone-dependent neoplastic disease in a mammal, the method comprising administering to a mammal in need thereof a pharmaceutically effective amount of a composition of claim 1.

15. The method according to claim 13, wherein the hormone-dependent neoplastic disease is selected from the group consisting of: uterine myometrial fibroids; endometriosis; benign prostatic hypertrophy; and carcinomas and adenocarcinomas of the endometrium, ovary, breast, colon, prostate, pituitary, and meningioma.

16. A method of synchronizing estrus in a mammal, the method comprising administering to a mammal in need thereof a pharmaceutically effective amount of a composition of claim 1.

17. A method of treating dysmenorrhea in a mammal, the method comprising administering to a mammal in need thereof a pharmaceutically effective amount of a composition of claim 1.

18. A method of treating dysfunctional uterine bleeding in a mammal, the method comprising administering to a mammal in need thereof a pharmaceutically effective amount of a composition of claim 1.

19. A method of inducing amenorrhea in a mammal, the method comprising administering to a mammal in need thereof a pharmaceutically effective amount of a composition of claim 1.

20. A method of treating to symptoms of premenstrual syndrome and premenstrual dysphoric disorder in a mammal, the method comprising administering to a mammal in need thereof a pharmaceutically effective amount of a composition of claim 1.

21. A method of contraception which comprises administering to a female of child bearing age for 28 consecutive days:
a) a first phase of from 14 to 24 daily dosage units of a progestational agent equal in progestational activity to about 35 to about 100 μg levonorgestrel; and
b) a second phase of from 1 to 11 daily dosage units, at a daily dosage of from about 2 to 50 mg, of an antiprogestin compound according to claim 1.

22. The method according to claim 21, wherein said medicament further comprises a third phase of daily dosage units of an orally and pharmaceutically acceptable placebo for the remaining days of the 28 consecutive days in which no antiprogestin, progestin or estrogen is administered.

23. The method according to claim 21, wherein the progestational agent is tanaproget.

24. The method according to claim 21, wherein the first phase further comprises co-administering an estrogen at a daily dose of 10 to 35 μg.

25. The method according to claim 21, wherein the second phase further comprises co-administering an estrogen at a daily dose of 10 to 35 μg.

26. The method according to claim 24, wherein the estrogen is ethinyl estradiol.

27. The method according to claim 25, wherein the estrogen is ethinyl estradiol.

28. The method according to claim 21, wherein the first phase comprises 18 to 24 days.

29. The method according to claim 21, wherein the first phase comprises 21 days.

30. The method according to claim 21, wherein the second phase comprises 3 days.

31. The method according to claim 22, wherein the third phase comprises 4 days.

32. A pharmaceutically useful kit adapted for daily oral administration which comprises:
a) 14 to 21 daily dosage units of a progestational agent equal in progestational activity from about 35 to about 150 μg levonorgestrel;
b) 1 to 11 daily dosage units of an antiprogestin compound of claim 1, each daily dosage unit containing an antiprogestin compound at a daily dosage of from about 2 to 50 mg; and
c) one or more packages for said daily dosage units.

33. The pharmaceutically useful kit according to claim 32, further comprising daily dosage units of an orally and pharmaceutically acceptable placebo, wherein the total daily dosage units in said kit is 28.
